(19) United States
(12) Patent Application Publication
(10) Pub. No.: US 2005/0209516 A1
(43) Pub. Date: Sep. 22, 2005

(54) Vital signs probe

(76) Inventor: Jacob Fraden, Ja Jolla, CA (US)

Correspondence Address: Jacob Fraden, Suite M, 6266 Ferris Sq., San Diego, CA, 92121, US

(21) Appl. No.: 10/806,766

(22) Filed: Mar. 22, 2004

(51) Int. Cl. A61B 5/00

(52) U.S. Cl. 600/323; 600/549

(57) ABSTRACT

A combination of a patient core temperature sensor and the dual-wavelength optical sensors in an ear probe or a body surface probe improves performance and allows for accurate computation of various vital signs from the photo-plethysmographic signal, such as arterial blood oxygenation (pulse oximetry), blood pressure, and others. A core body temperature is measured by two sensors, where the first contact sensor positioned on a resilient ear plug and the second sensor is on the external portion of the probe. The ear plug changes it's geometry after being inserted into an ear canal and compress both the first temperature sensor and the optical assembly against ear canal walls. The second temperature sensor provides a reference signal to a heater that is warmed up close to the body core temperature. The heater is connected to a common heat equalizer for the temperature sensor and the pulse oximeter. Temperature of the heat equalizer enhances the tissue perfusion to improve the optical sensors response. A pilot light is conducted to the ear canal via a contact illuminator, while a light transparent ear plug conducts the reflected lights back to the light detector.

### FIELD OF INVENTION

This invention relates to devices for monitoring physiological variables of a patient and in particular to a device for monitoring arterial pulse oximetry and temperature from an ear canal. This invention is based on the provisional patent application Ser. Nos. 60/449,113 and 60/453,192.

### DESCRIPTION OF PRIOR ART

Monitoring of vital signs continuously, rather than intermittently is important at various locations of a hospital—in the operating, critical care, recovery rooms, pediatric departments, general floor. etc. If accuracy is not compromised, the preference is always given to non-invasive methods as opposed to invasive. Also, a preference is given to a device that can provide multiple types of vital signs instead of receiving such information from many individual sensing devices attached to the patient. Just a mere packaging of various sensors in a single housing typically is not efficient for the following reasons: various sensors may require different body sites, different sensors may interfere with each other functionality, a combined packaging may be more susceptible to motion and other artifacts and the size and cost may be prohibiting.

An example of a combined vital signs sensor is U.S. Pat. No. 5,673,692 issued to Schultze et al. where an ear infrared temperature sensing assembly (a tympanic thermometer) is combined with a blood pulse oximeter. While an ear is an excellent location for the temperature monitoring and an infrared probe may be very accurate when used intermittently, it doesn't lend itself to a continuous monitoring due to its strong sensitivity to a correct placement, motion artifacts, and adverse effects of the ear canal temperature on the infrared sensing assembly. A device covered by U.S. patent application Ser. No. 09/927,179 filed on Aug. 8, 2001, offers a better way for a continuous monitoring of the body core temperature through the ear canal. It is based on a contact (non-infrared) method where a temperature gradient is measured across the ear canal and the external heater brings this gradient to a minimal value. As a result, the heater temperature becomes close to that of an internal body (core) temperature.

Concerning other vital signs that potentially can be monitored through an ear canal, an arterial pulse oximetry is a good candidate as demonstrated by the above mentioned patent issued to Schultze et al. Yet, presence of an infrared optical system in the ear canal results in extremely high motion artifacts during even minimal patient movements. Another problem associated with monitoring blood oxygenation through the ear canal is a relatively low blood perfusion of the ear canal lining. A good method of improving blood perfusion is to elevate temperature of the oximeter sensing device, as exemplified by U.S. Pat. No. 6,466,808 issued to Chin et al.

The degree of oxygen saturation of hemoglobin, SpO₂, in arterial blood is often a vital index of a medical condition of a patient. As blood is pulsed through the lungs by the heart action, a certain percentage of the deoxyhemoglobin, RHb, picks up oxygen so as to become oxyhemoglobin, HbO₂. From the lungs, the blood passes through the arterial system until it reaches the capillaries at which point a portion of the HbO₂ gives up its oxygen to support the life processes in adjacent cells.

By medical definition, the oxygen saturation level is the percentage of HbO₂ divided by the total hemoglobin. Therefore,

$${Sp O}_{2} = \frac{{Hb O}_{2}}{RHb + {Hb O}_{2}} \qquad ( 1 )$$

The saturation value is a very important physiological number. A healthy conscious person will have an oxygen saturation of approximately 96 to 98%. A person can lose consciousness or suffer permanent brain damage if that person's oxygen saturation value falls to very low levels for extended periods of time. Because of the importance of the oxygen saturation value pulse oximetry has been recommended as a standard of care for every general anesthetic.

The pulse oximetry works as follows. An oximeter determines the saturation value by analyzing the change in color of the blood. When radiant energy interacts with a liquid, certain wavelengths may be selectively absorbed by particles which are dissolved therein. For a given path length that the light traverses through the liquid, Beer's law (the Beer-Lambert or Bouguer-Beer relation) indicates that the relative reduction in radiation power (P/Po) at a given wavelength is an inverse logarithmic function of the concentration of the solute in the liquid that absorbs that wavelength.

In general, methods for noninvasively measuring oxygen saturation in arterial blood utilize the relative difference between the electromagnetic radiation absorption coefficient of deoxyhemoglobin, RHb, and that of oxyhemoglobin, HbO₂. The electromagnetic radiation absorption coefficients of RHb and HbO₂ are characteristically tied to the wavelength of the electromagnetic radiation traveling through them.

A standard method of monitoring non-invasively oxygen saturation of hemoglobin in the arterial blood is based on a ratiometric measurement of absorption of two wavelengths of light. One wavelength is in the infrared spectral range (typically from 805 to 940 nm) and the other is in red (typically between 650 and 750 nm). Other wavelengths, for example in the green spectral range, are used occasionally as taught by U.S. Pat. No. 5,830,137 issued to Scharf.

In its standard form, pulse oximetry is used in the following manner: the infrared and red lights are emitted by two light emitting diodes (LEDs) placed at one side of a finger clamp or an ear lobe. The signals from each of the wavelengths ranges are detected by a photodiode at the opposing side of the ear lobe or at the same side of a finger clamp after trans-illumination through the living tissue perfused with arterial blood. Separation of the signals from the two wavelength bands is performed by alternating the current drive to the respective light emitting diode (time division), and by use of the time windows in the detector circuitry or software. Both the static signal, representing the intensity of the transmitted light through the finger or ear lobe and the signal synchronous to the heart beat, i.e., the signal component caused by the artery flow, is being monitored.

One problem that is associated with use of a pulse oximetry sensor on a digit (a finger or toe) or an extremity (ear lobe or helix, e.g.) or even on the body surface is a sensitivity to patient movements and effects of ambient light. Numerous methods of data processing have been proposed to minimize motion artifacts. Yet, obviously the best method would be to place a probe at such a body site that is much less affected by the patient movement and is naturally shielded from the ambient illumination so there will be easier to counteract the smaller artifacts. The above mentioned U.S. Pat. No. 5,673,692 describes a pulse oximeter sensor installed into an ear canal probe. This indeed is a move in a right direction. However, the design has all optical components positioned inside the ear canal and that my not lend itself to a practical and cost-effective device.

Another important vital sign that needs to be non-invasively continuously monitored is arterial blood pressure. While a direct blood pressure can be continuously monitored by invasive catheters, the indirect blood pressure can be measured with help of an inflating cuff positioned over a limb or finger, or alternatively, by computing blood pressure from the pulsatile arterial blood volume. The last method is based on a plethysmography which can be either electro-plethysmography (EPG) which measures tissue electrical resistance or photo-plethysmography (PPG) which measures the tissue optical density. The plethysmography in combination with an electrocardiographic (EKG) wave can yield a number that is related to the arterial blood pressure (see for example K. Meigas et al. Continuous Blood Pressure monitoring Using Pulse Delay. Proc. of 23^{rd} Annual EMBS International Conf. 2001, Oct. 25-28, Istanbul). It should be noted that PPG and pulse oximetry are based on the same type of a sensor—a combination of a light emitting device and light sensing device.

Thus, it is a goal of this invention to provide a combined sensing assembly for various physiological variables that is less sensitive to motion artifacts;

It is another goal of this invention to provide an blood pulse oximetry probe suitable for placement inside the ear canal;

It is also a goal of this invention to provide an accurate vital sign probe for the ear canal to provide continuous monitoring of pulse oximetry and body core temperature;

It is also a goal of the invention to provide a combined sensing assembly that can collect information on blood oxygenation along with body core temperature.

And another goal of the invention is provide an ear probe that can be used for indirect measurement of arterial blood pressure.

### SUMMARY OF INVENTION

A combination of a patient core temperature sensor and the dual-wavelength optical sensors in an ear probe or a body surface probe improves performance and allows for accurate computation of various vital signs from the photo-plethysmographic signal, such as arterial blood oxygenation (pulse oximetry), blood pressure, and others. A core body temperature is measured by two sensors, where the first contact sensor positioned on a resilient ear plug and the second sensor is on the external portion of the probe. The ear plug changes it's geometry after being inserted into an ear canal and compress both the first temperature sensor and the optical assembly against ear canal walls. The second temperature sensor provides a reference signal to a heater that is warmed up close to the body core temperature. The heater is connected to a common heat equalizer for the temperature sensor and the pulse oximeter. Temperature of the heat equalizer enhances the tissue perfusion to improve the optical sensors response. A pilot light is conducted to the ear canal via a contact illuminator, while a light transparent ear plug conducts the reflected lights back to the light detector.

### BRIEF DESCRIPTION OF DRAWINGS

FIG. 1 is a general view of the combined sensing assembly with a rigid optical extension positioned inside the ear canal

FIG. 2 shows insertion of the ear plug into the sensing head

FIG. 3 is the cut out view of the sensing head with the ear plug attached

FIG. 4 depicts positions of the light emitting diodes in a rigid extension

FIG. 5 is a block diagram of the sensing device with thermocouple sensors

FIG. 6 is a general view of the pulse oximetry probe positioned inside the ear canal

FIG. 7 shows a cut-out view of the probe and the ear sensing plug in a disconnected position

FIG. 8 is a block diagram of the ear canal pulse oximeter

FIG. 9 depicts the cut-out view of the probe with an illuminator permanently attached to the probe

FIG. 10 is the cut-out view of the sensing assembly positioned inside the ear canal

FIG. 11 is a cross-sectional view of the optical sensor with a separated ear plug

FIG. 12 is a frontal view of the optical/temperature sensor

FIG. 13 is a cross-sectional view of the probe with a dual ear plug.

FIG. 14 shows a combination sensor for skin application

FIG. 15 is a cross-sectional view of the skin sensor with a disposable sensing cup

FIG. 16 is shows a time dependence of the temperature detectors

FIG. 17 depict combination of infrared and red PPG waves

FIG. 18 shows variations in the decaying slope of the PPG wave

FIG. 19 illustrates a combination of EKG and PPG waves

FIG. 20 shows arterial pressure as function of time delay.

### DESCRIPTION OF PREFERRED EMBODIMENTS

The present invention provides for an optical photo-plethysmographic assembly for an ear canal. The assembly can be further supplemented by the deep body temperature monitoring components. These components will improve quality of the photo-plethysmographic signals received from the optical assembly positioned inside the ear canal. A combined sensor has an improved performance as compared with the separately used devices. The invention solves two major issues related to placing a pulse oximetry sensor inside the ear canal. The first issue is a secure positioning that would minimize motion artifacts. The second issue is an improved blood perfusion of the earl canal lining, thus enhancing the detected signal. There are several embodiments of the invention. Each embodiment has its own advantages and limitations. The most important embodiments are described in detail below.

First Embodiment

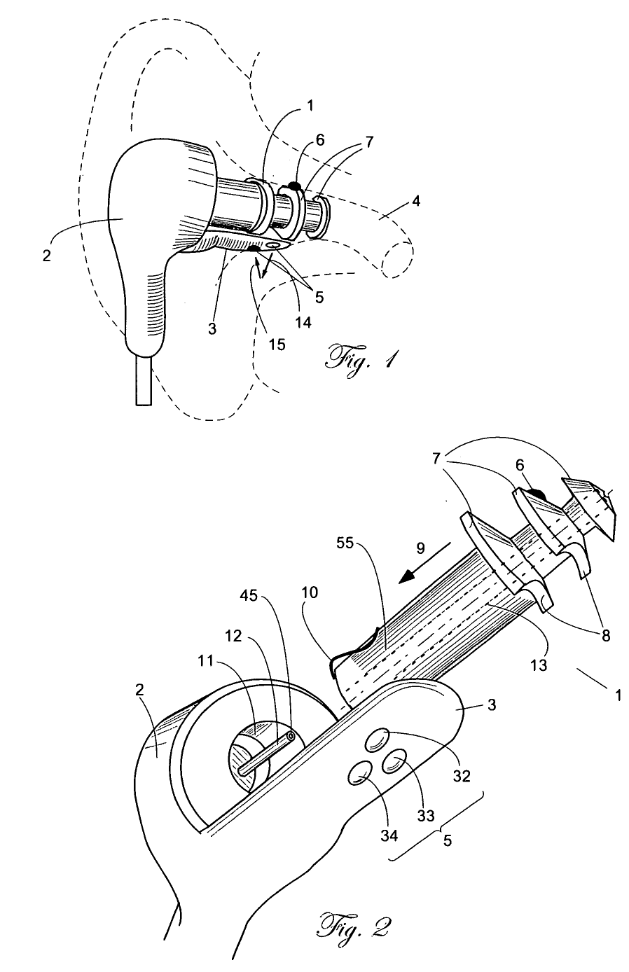

FIG. 1 shows plug 1 attached to ear probe 2. Probe 2 has a sensing extension 3 that carries blood oximetry windows 5. Plug 1 is fabricated of plaint, flexible and resilient material, such as silicone. A compressible foam also may be used.

Before the vital signs monitoring starts, plug 1 and extension 3 are inserted together into ear canal 4. This combination of extension 3 and a resilient ear plug 1 allows for a secure and stable positioning of the optical windows 5 against ear canal 4 walls. Extension 3 may be either rigid or somewhat flexible to accommodate variations of the ear canal shapes, while ear plug 1 is acting like a spring conforming its own contour to the ear canal shape and applying pressure on extension 3, pushing it against the ear canal wall. It should be appreciated that plug 1 has somewhat different shapes before, during and after insertion into the ear canal. Its original shape (before insertion) may have many configurations. However, it appears that a shape with one or more extended ribs 7 (see also FIG. 2) provides a good spring action. Windows 5 typically consist of three windows (only two are visible in FIG. 1). Two of them emit light rays 14 from first and second windows 32 and 33 and one receives reflected rays 15 through a third window 34 as in FIG. 2. This assembly contains all components required for obtaining the photo-plethysmographic signals for further data processing to compute the arterial blood oxygenation, arterial pressure, etc.

To improve functionality of the probe by means of a temperature measurement function, plug 1 carries on or near its outer surface temperature sensor 6. That sensor is in an intimate thermal contact with ear canal 4 walls. Temperature sensor 6 may be positioned on extension 3 (not shown) near windows 5. In that case, extension 3 should be fabricated of a material with low thermal conductivity, meaning that it should be thermally de-coupled from probe 2. Alternatively, temperature sensor 6 may be position on plug 1 at the opposite side from extension 3 as in FIGS. 1 and 2. Plug 1 may be plugged into probe 2 as shown in FIG. 2 where it moves in direction 9 along extension 3 until its lower portion 55 is inserted into receptacle 11. Plug 1 may have an internal hollow channel 13 that is placed over pin 12. When temperature sensor 6 is carried by one of the ribs 7, its two terminal wires are passing through the body of plug 1. One wire 10 is shown in FIG. 2. Upon insertion into probe 2, wire 10 makes electrical contact with a conductive wall of receptacle 11. The other wire (not shown) may be positioned inside channel 13 to make electrical contact with pin 12. To accommodate for the shape of extension 3, ribs 7 may have cut-outs 8. Pin 12 may be hollow with bore 45 passing though the entire probe 2 to the open atmosphere. This bore in combination with channel 13 allows for air pressure equalization between the ear canal interior and the outside.

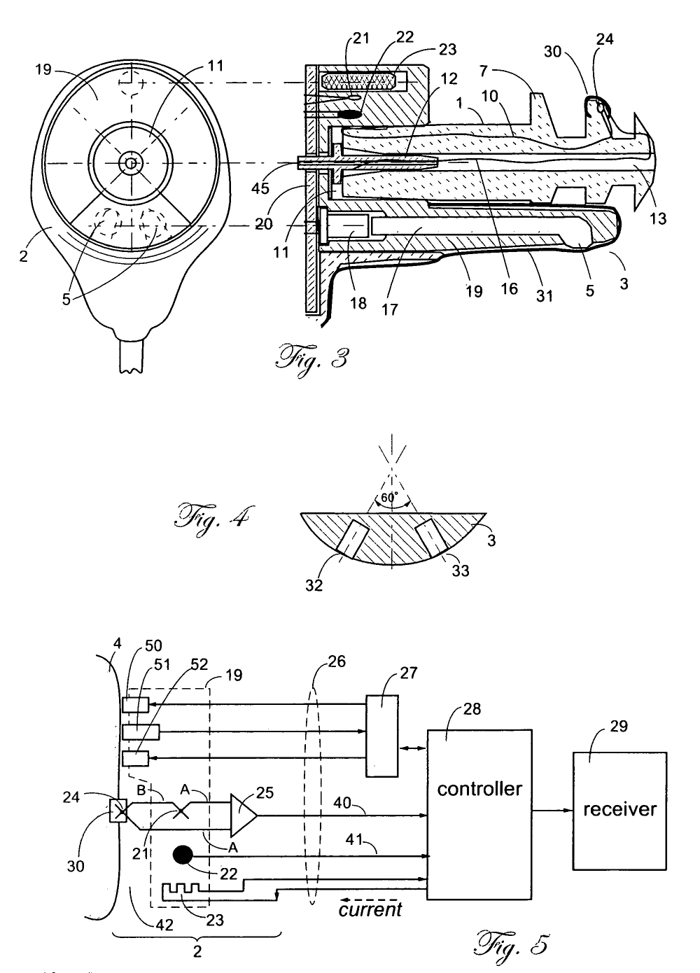

FIG. 3 further illustrates positions of various components in probe 2. The left side image is the front view of probe 2 without plug 1, while the right side image is a cross-sectional view of the assembly with plug 1 inserted into receptacle 11. Wires 10 and 16 make the respective electrical contacts with walls of receptacle 11 and pin 12. In turn, receptacle 11 and pin 12 make contacts with circuit board 20.

Wires 10 and 16 may be dissimilar metals A and B forming first thermocouple junction 24. To improve thermal contact with the ear canal 4 walls, the junction is thermally connected to an intermediate metal button 30 which may be fabricated of brass or other heat conducting material. Wires 10 and 16 eventually make electrical contacts with the printed circuit board 20 that carries the second thermocouple junction 21 (also metals A and B) incorporated into heat equalizer 19. One should not be limited with use of the thermocouple temperature sensor. Equally effective may be the thermistor or any other conventional temperature detector.

Note that wires of the same type (A in this example) make electrical connection to electronic components, such as pre-amplifier 25 in FIG. 5. The same heat equalizer also carries temperature sensor 22 and, through its portion that is a part of extension 3, it also carries light guides 17 and detector/emitters 18 (only one of each is shown in FIG. 3). Heat equalizer 19 is fabricated of metal having good thermal conductivity, such a aluminum, copper, zinc or other appropriate metal. Light guides 17 are terminated with windows 5 (only one is shown in FIG. 3). For the sanitary purposes, extension 3 and portion of probe 2 may be covered with a disposable probe cover 31. The probe cover may be fabricated of such material as polypropylene having thickness ranging from 0.0005 to 0.010″ and having an appropriate conforming shape to envelop components that may come in contact with the patient's tissues.

First, we describe operation of the temperature measurement components. Considering FIGS. 3 and 5 note that thermocouple junctions 24 and 21 provide electric signal that is nearly proportional to a temperature gradient Δ between button 30 and heat equalizer 19. That signal is amplified by pre-amplifier 25 and channeled out of the probe via a communication link, for example cable 26. The absolute temperature Tₐ of heat equalizer 19 is measured by an imbedded temperature sensor 22, for example a thermistor. Thus, temperature sensor 22 also measures temperature of second thermocouple junction 21. The internal core (deep body) temperature T_{b} can be computed from an equation that accounts for the temperature gradient Δ.
T_{b}=Tₐ+(1+μ)Δ (2)
where value of is not constant but is function of both Tₐ and T_{b}. Its functional relationships shall be determined experimentally.

To further improve accuracy, value of Δ should be minimized. This can be achieved by adding a heater to heat equalizer 19. Pre-amplifier's 25 output signal 40 representing Δ and temperature signal 41 from temperature sensor 22 pass to controller 28 that provides electric power to heater 23 imbedded into heat equalizer 19. Controller 28 regulates heater in such a manner as to minimize temperature difference Δ, preferably close to zero. Since button 30 that carries first junction 24 is attached to a wall of ear canal 4, temperature of heat equalizer 19 eventually becomes close to that of ear canal 4. After some relatively short time (few minutes) ear canal walls assume the inner temperature of the patient body. It is important, however that first 24 and second 21 thermocouple junctions are thermally separated from each other by some media 42 of low thermal conductivity. Plug 1 being fabricated of low heat conducting resin, for example silicone rubber, acts as such media. Temperature Tₐ of heat equalizer 19 becomes close to the patient inner body core temperature T_{b}.

Extension 3 that carries three windows 32, 33, and 34 (FIG. 2) provides the photo-plethysmographic sensing function. Light guide 17 (FIG. 3) is optically connected to detectors/emitters 18. There are three light guides 17 in extension 3 and detector/emitters 18, but only one is shown for clarity. Alternatively, detector/emitters 18 may be positioned next to windows 5 thus eliminating a need for light guides 17. Detector/emitters 18 contain one of the following (see also FIG. 5): first light emitting diode (LED) 50 operating at visible wavelength of about 660 nm, second LED 52 operating at near infrared wavelength of about 910 nm, and light detector 51 covering both of the indicated wavelengths. Light guides 17 should be fabricated of material with low absorption in the wavelengths of operation. Examples of the materials are glass and polycarbonate resin. Windows 32 and 33 preferably should be aimed along axes forming an approximate 60° angle to each other (FIG. 4). Window 34 (not shown in FIG. 4) should form an angle of about 30° to each of them. All these components form an optical head of a pulse oximeter. It detects the photo-plethysmographic waves of the pulsatile blood at two wavelengths and pass them to module 27 for the signal processing.

There are many possible versions of operating LEDs 50, 52 and detector 51 and analyzing the photo-plethysmographic waves that allow computation of the oxygen saturation of hemoglobin in arterial blood. These methods are well known in art of pulse oximetry and thus not described here. Yet, an important contribution from the temperature side of probe 2 is that heat equalizer 19 elevates temperature Tₐ of extension 3 to the level that is close to a body core temperature. This increases blood perfusion in the ear canal walls that, in turn, improves signal-to-noise ratio of a photo-plethysmographic pulse.

It should be noted, that just a mere elevation of temperature of the pulse oximetry components may improve blood perfusion and enhance accuracy. The elevation may be few degrees less or more than the core temperature. Therefore, temperature sensor 6 may be absent while heater 23 and sensor 22 would keep temperature of the assembly above ambient and preferably close to the patient's body, say 37° C. Signals from a pulse oximeter module 27 and temperature controller 28 pass to receiver 29 that may be a vital sign monitor or data recorder. Naturally, a communication link that in FIG. 5 is shown as cable 26 can be of many conventional designs, such radio, infrared or

Second Embodiment

In this embodiment, photons of light that are modulated by the pulsatile blood to produce the photo-plethysmographic signals pass through a translucent ear plug. Thus, the essential component of this embodiment is a light transparent ear plug that also may be used as a carrier of a temperature sensor. Contrary to the first embodiment, when the optical components were incorporated into extension 3, the ability of an ear plug to transmit light allows to keep most of the optical components outside of the ear canal and thus simplifies design and use of the device.

Since the pulse oximetry data and indirect blood pressure monitoring can be accomplished from signals that are measured by the same optical probe, the same components that are used for the ear pulse oximetry are fully applicable for the indirect arterial blood pressure monitoring as well.

The light emitting devices (for example, light emitting diodes—LED) are positioned inside probe 62 (FIG. 6) that is positioned outside of the patient body, while only ear plug 64 is inserted into ear canal 4 of ear 60. Illuminator 65 is adjacent to the entrance of the ear canal and shielded by shield 66 from a direct optical coupling with ear plug 64. Thus, light transmission assembly 63 is comprised of illuminator 65, shield 66 and ear plug 64. Illuminator 65 and ear plug 64 should be substantially optically homogeneous and transparent in the wavelengths of the lights emitted by the LEDs. Yet, they not necessarily need to be fabricated of the same material. For example, illuminator 65 may be fabricated of acrylic resin while ear plug 64 may be fabricated of clear silicone resin. It may be desirable, however, that the illuminator has certain flexibility and pliability for better conformation to and coupling with the ear canal entrance. Shield 66 may be fabricated of any material that is opaque for the used light. Each of these components (illuminator, shield and plug) may be either reusable or disposable.

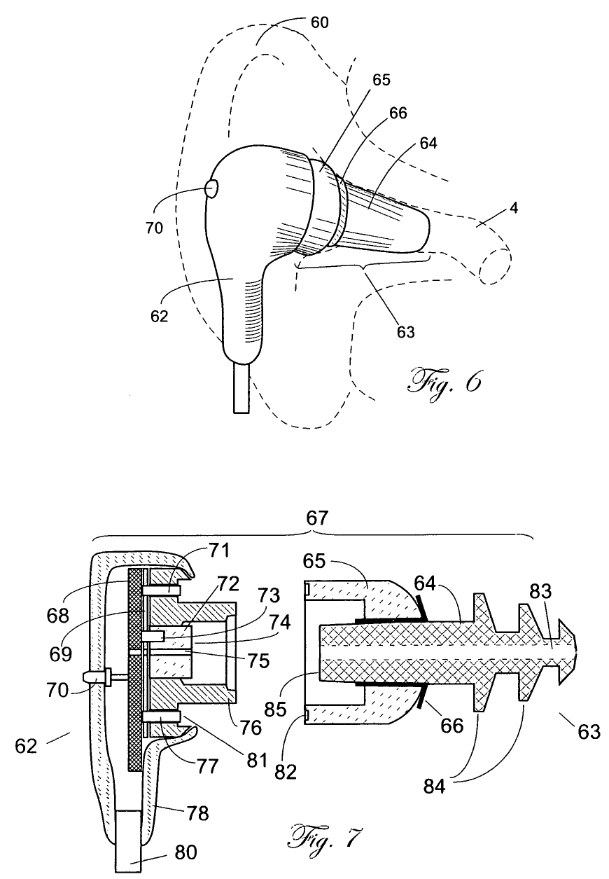

FIG. 7 illustrates the internal structure of oximetry sensor 67 where light transmission assembly 63 is disconnected from probe 62. This ability to disconnect may be important for practical use as the entire light transmission assembly 63 may be made interchangeable and even (disposable. The probe 62 internal components are protected from the environment by encapsulation 78 and data are transmitted via cable 80. However, data may be transmitted by other means, for example via radio or optical communication links. Internal circuit board 68 supports holder 76, light coupler 72, two LEDs 71 and 77, light detector 73 and heart rate indicating light 70. Heater 69 may be added to warm up the interior of probe 62 and portion of ear plug 64 to temperatures in the range of 37-40° C. which would aid in increasing blood perusing in the ear canal and, as a result, enhance a magnitude of the detected signal. Positions of the light emitting and detecting components may be reversed if so desired for a particular design. That is, an “illuminator” may contain a detector and the ear plug may be coupled with the emitters. This arrangement will not change the general operation of the device.

Light transmitting assembly 63 may be plugged into holder 76 so that butt 85, which is part of ear plug 64, comes in proximity with end 74 of light coupler 72. This would allow light to pass from the body of ear plug 64 via its butt 85 and light coupler 72 toward light detector 73. At the same time, illuminator 65 has at its end joint 82 that comes in proximity with lens 81 of second LED 77. The same is true for first LED 71. Thus, after installation of light transmission assembly 63 onto holder 76, both LEDs can send light through illuminator 65. As in many conventional pulse oximeters. LEDs can operate with a time division of light transmission to prevent sending two wavelengths at the same time. Note that shield 66 prevents light of any wavelength from going directly from illuminator 65 toward ear plug 64. Since ear plug 64 is intended for insertion into an ear canal, to aid in this function, hollow bore 83 may be formed inside ear plug 64. Similar hole 75 (or other air passing channel) is formed in light coupler 72 and other components of probe 62 to vent air to the atmosphere. The bore and a hole will allow for air pressure equalization when ear plug is inserted into an ear canal. Alternatively, the bore may be replaced with a groove positioned on the exterior of ear plug 64 (not shown).

While FIG. 6 shows ear plug 64 having a smooth surface, FIG. 7 shows a variant of ear plug 64 with protruding ribs 84 that are pliable, flexible and resilient. As seen in FIG. 10, when ear plug 64 is inserted into ear canal 4, ribs 84 flex and secure the plug inside the ear canal. While ear plug 64 may be rigid, it is more advantageous to have it flexible, pliant and resilient, so that it would conform to the shape of the ear canal.

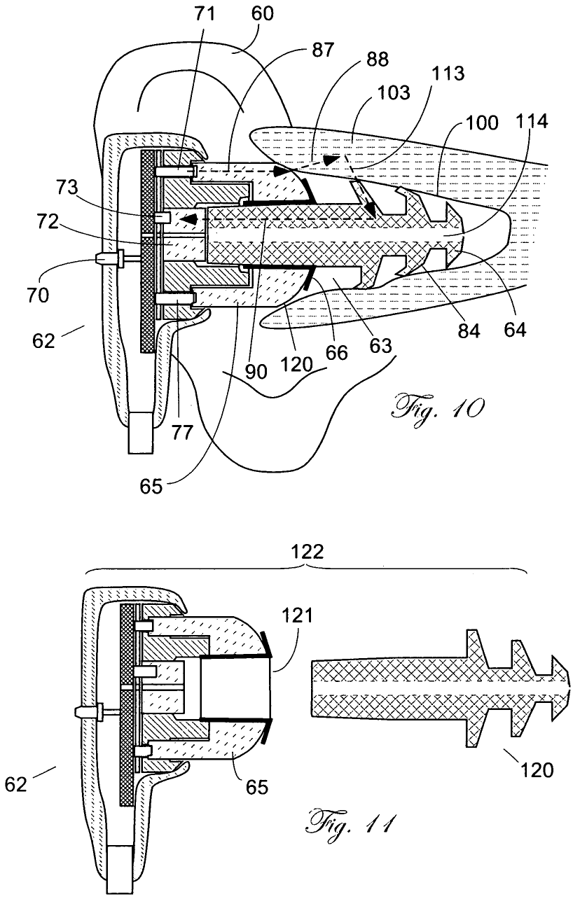

It should be noted that the purpose of illuminator 65, light transmissive ear plug 64 and shield 66 is to separate the transmissive and receiving beams of light. Otherwise, the transmissive light would spuriously couple directly to light detector 73, thus bypassing biological tissue 103. There are many possible ways of separating the transmitting and receiving beams of light, but all involve the use of a light transparent ear plug. As an illustration of another possible design, FIG. 14 shows dual ear plug 104, consisting of two light transmitting sections—first section 108 and second section 110. These sections are separated by light stopper 109 that is not transparent for the used wavelengths of light. First and second LEDs (71 and 77) are coupled to first section 108, while detector 73 is coupled to second section 110 by means of the intermediate light conducting rod 106. Two LEDs (71 and 77) produce light in form of transmitting beam 112 that propagates toward tissue 103 and modulated by oxyhemoglobin. The modulated light in form of receiving light beam 111 passes toward detector 73. The separation of the light beams are performed by light stopper 109 and jacket 105 which is also opaque. Naturally, in this case there is no need for a separate illuminator as both transmission and reception of light is performed by different sections of the ear plug.

The entire sensing assembly works as follows (see FIG. 10). First LED 71 emits light that in form of first beam 87 travels through the body of illuminator 65 which comes in physical contact 120 with the opening of the ear canal. This contact allows light (in form of second beam 88) to continue traveling into the biological tissue and be modulated by the oxyhemoglobin and pulsatile blood volume. The scattered and modulated light (in form of third beam 113) enters the body of ear plug 64 and propagates toward light detector 73 in form of fourth beam 90. The identical process is true for the light emitted by second LED 77 when it is activated, in turn. Both detected signals from the same detector 73 are processed in a conventional way to obtain information on blood oxygenation, blood pressure and hear rate. Each detected heart beat can activate light 70 to provide a visual feedback to an operator on a functionality of the device and patient's heart activity. Since plug 64 is secured inside ear canal and illuminator 65 has large contact area and is pressed against ear canal opening, motion artifacts are reduced significantly. Also, spurious ambient light is shielded from the ear canal interior by a scull and is not affecting signals detected by detector 73.

While FIG. 7 shows light transmission assembly 63 as a component that may be removed, FIG. 11 demonstrates that a removable and preferably disposable unit 120 may contain just ear plug 64 while illuminator 65 is a permanent part of probe 62. Before placing into an ear canal, disposable unit 120 is inserted into opening 121 in illuminator 65 and shield 66 to form a complete assembly 122 that is used for sensing.

FIG. 8 depicts a general block diagram of an ear canal pulse oximeter and/or blood pressure monitor. The returned modulated light in form of fourth beam 90 is received by detector 73 that is connected to amplifier 91. Alternating light emissions by LEDs 71 and 77 are activated by controller 92 as well as gating the corresponding response of amplifier 91. Controller 92 feeds detected and amplified signals to processor 93 that makes all necessary computations and sends signal to monitor 94. There may be numerous additional components in the device, like a power supply, radio communication channel, an alarm, etc., however, they are of conventional designs and not subject of this invention. FIG. 18 illustrates two PPG waves, infrared 203 and red 204. These waves are derived from detector 73 by subtracting a background (baseline) signals by processor 93. Blood oxygen saturation may be computed from an experimental formula:
SpO₂=110−25X, (3)
where X is ratio of the red and infrared wave amplitudes.

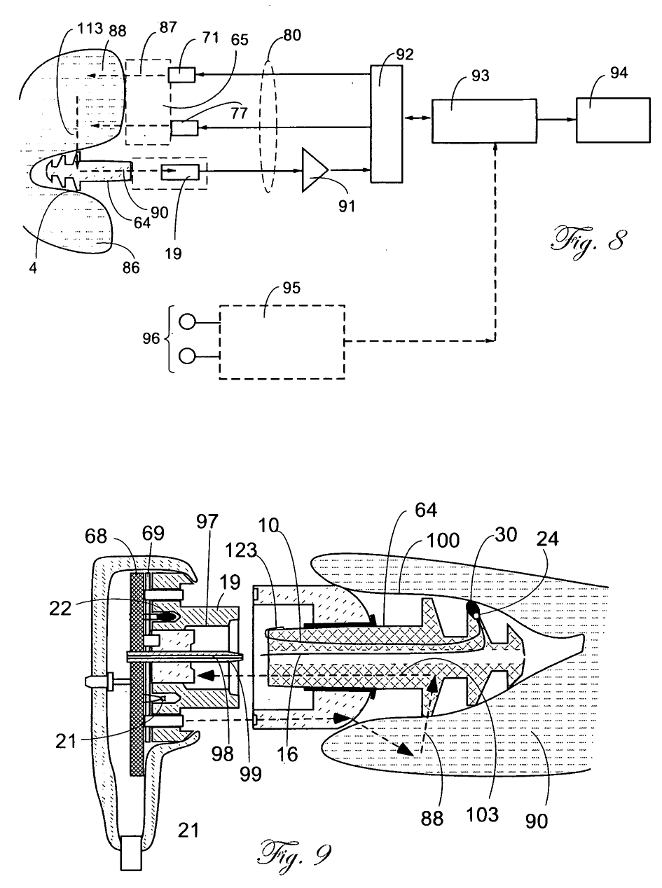

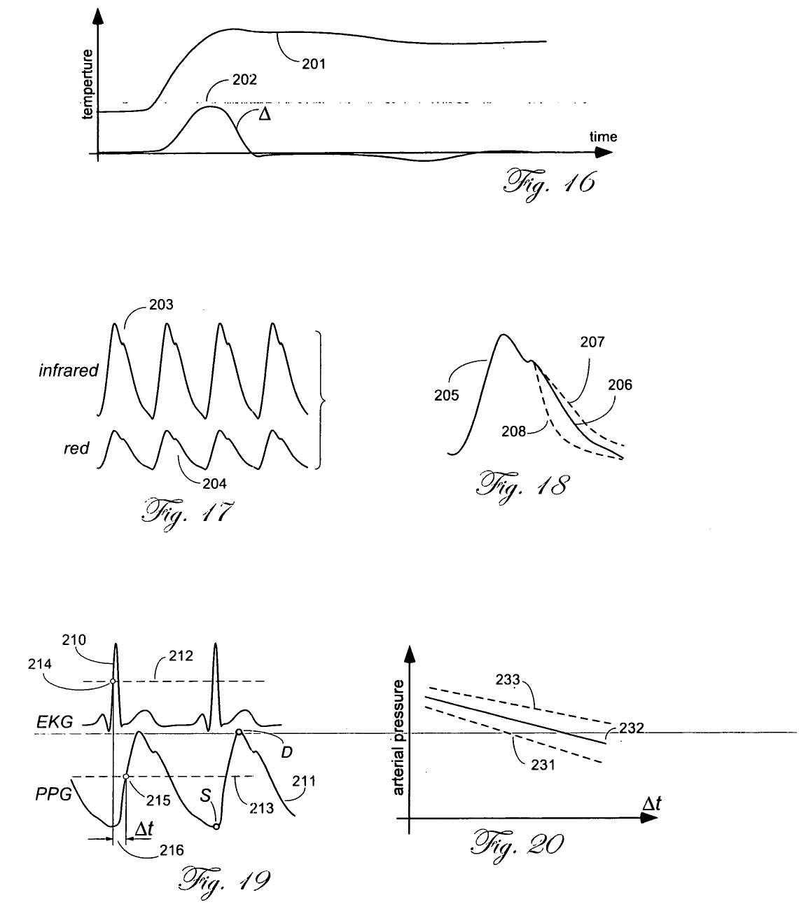

FIG. 9 shows that the core temperature can be monitored in a way similar to that shown in FIGS. 3 and 5. A thermocouple temperature sensor is formed by two dissimilar wires 10 and 16. Cold junction 21 (a reference) is connected to circuit board 68 and is imbedded into heat equalizer 19. Naturally, a thermocouple temperature sensor can be replaced with any type of temperate detector, like a thermistor, semiconductor, etc. The sensor type makes no difference for the overall performance as long as the basic functionality is preserved. The heat equalizer is a good thermal conductor and preferably should be fabricated of aluminum, copper or other appropriate metal. The thermocouple dissimilar wires 10 and 16 (for example, iron and constantan) are imbedded into ear plug 64 along its length. To operate, they must be electrically connected to cold junction 21. For first wire 10, this is accomplished by its electrical connection to heat equalizer 19 that in turn has electrical contact on circuit board 68. Bend 123 of wire 10 aids in making a good electrical contact. Second wire 16 is connected to circuit board 68 by touching pin 99 which may have hollow canal 98 to equalize ear and atmospheric pressures. Pin 99 is fabricated of electrically conductive material. Temperature of heat equalizer 19 is measured by an absolute temperature sensor, for example an imbedded temperature sensor 22 which may be a thermistor. It should be appreciated that in a normal operation, temperatures of heat equalizer 19, pin 99, thermistor temperature sensor 22 and cold junction 21 are nearly equal. Heater 69 warms up the entire assembly to such temperature as to minimize a thermal gradient between hot junction 24 and cold junction 21. The device operation is similar that that described above with respect to FIGS. 3 and 5. FIG. 16 illustrates how temperature 201 of thermistor 22 changes with operation of the heater. It also shows temperature difference 202 (Δ) from thermocouple junctions 24 and 21. Note that Δ is brought to zero and the thermistor warms up to the current patient temperature.

To take full benefits of the present invention, the thermal and optical components in a probe should be located in close proximity to each other. FIG. 12 illustrates how these components may be mutually positioned. Note, that for better signal-to-nose ratio, more than one LED can be used for each wavelength, that is, two LEDs 71a and 71b are used for red and 77a and 77b are used for the infrared light. The identical LEDs should be positioned at the opposite sides of the probe, while cold junction 21 and thermistor 22 can be positioned in-between.

Third Embodiment

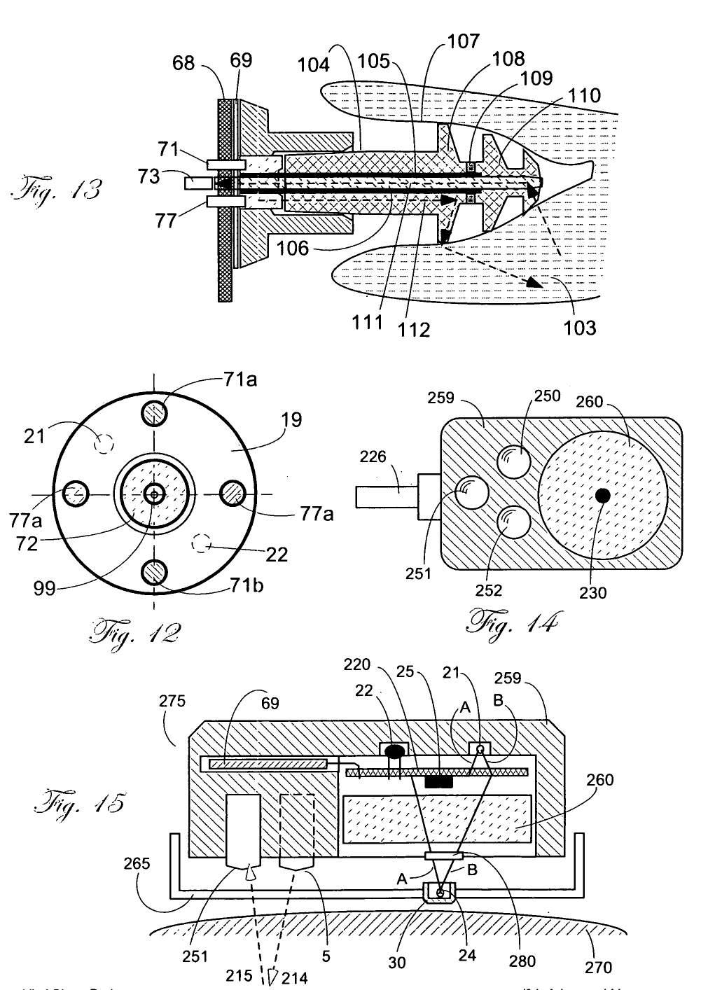

The above described sensing assemblies can be modified for use on an outside surface of a patient body, preferably above a bone, such as a scull or rib. FIG. 14 depicts a front plate that is to be placed on the patient skin. Like in the ear probe, it contains all essential components, such as heat equalizer 259 (analogous to equalizer 19), button 30, windows 250, 151 and 252, heater 69, cable 226. Thermal insulator 260 serves the same thermal function as probe 64 of FIG. 9. Insulator 260 may be made of polymer foam or it may be just a void inside the body of probe 275. The interior of the skin sensor is shown in FIG. 15 where first thermocouple junction 24 is positioned inside button 30 that makes intimate thermal contact with patient's skin 270. The button may be permanently attached to insulator 260, or alternatively, as shown in FIG. 15, it may be positioned on a disposable protective cup 265. That cup may be made of such material as polypropylene and may have an adhesive layer on the side facing skin 270. At least a portion of cup 265 that is adjacent to windows 250, 251 and 252 should be transparent for the employed wavelengths. Thermocouple wires A and B are attached to the circuit board 220 that also may carry pre-amplifier 25. It should be noted that instead of the thermocouple wires A and B, a thermistor or other type of a temperature sensor may be used to measure the skin surface temperature. This in no way would change the overall operation of the device. This statement applies to both the ear and the skin surface versions of the device.

Heater 69 is common for both the temperature sensing components (right side of FIG. 15) and the pulse-oximetry components (lefts side of FIG. 15). Heat equalizer 259 is warmed up to temperature Tₐ that is close to the body core temperature T_{b}. Thermocouple wires that form first junction 24 are shown as attached to circuit board 220. Additional thermocouple wire connector 280 may be used to allow separation of cup 265 from body of probe 275.

Computation of Blood Pressure

Since red and infrared signals from detector 19 produce identical shapes of PPG waves as shown in FIG. 17, one or both waves may be used for computing arterial blood pressure by processor 93. In a particular application where blood pressure is required but pulse oximetry is not monitored, only one light emitting device (LED) is needed for monitoring arterial blood pressure. FIG. 18 illustrates that a decaying slope of a PPG wave can have a slow decay 207, normal decay 206 or fast decay 208. The decay rate is related to a peripheral vascular resistance and, subsequently, to an arterial blood pressure. Thus an experimental relationship between the decay rate and blood pressure can be used for the latter computation. It is, however, may be necessary to calibrate the relationship to each individual patient. Another method of computing the arterial blood pressure is based on measuring time delay Δt between the EKG and PPG waves, as shown in FIG. 19. Naturally, the EKG waves need to be obtained from the electrodes placed on the patient body. FIG. 8 illustrates a pair of EKG electrodes 96 and EKG circuit 95 that feeds the EKG signal into processor 93. In processing, EKG wave 210 and PPG wave 211 cross respective thresholds 212 and 218. The cross-over points 214 and 215 are separated by time 216 which is delay Δt. It is an experimental fact that this time delay is inversely proportional to the mean blood pressure 232 as shown in FIG. 20. The diastolic 231 and systolic 233 pressures can be computed by using the spread between points D and S of the PPG wave (FIG. 13) as a scaling factor.

While the above description contains many specifics, these specifics should not be construed as limitations on the scope of the invention, but merely as exemplifications of preferred embodiments thereof. Those skilled in the art will envision many other possible variations that are within the scope and spirit of the invention.

1. A system for detecting photo-plethysmographic signals from a patient ear canal, comprising a sensor's housing, a first light emitting source, a light detector and a housing extension, wherein distal side of said housing extension is inserted into the patient ear and proximal side of the extension is optically coupled to said light emitting sources and light detector.

2. A system for detecting photo-plethysmographic signals from a patient ear canal as defined in claim 1 further comprising a second light emitting source operating at a different wavelength from first light source and a processor for computer arterial blood oxygenation.

3. A system for detecting photo-plethysmographic signals from a patient ear canal as defined in claim 1 wherein said housing extension is fabricated of material that is substantially transparent for wavelength of light generated by said first light emitting source.

4. A method for monitoring patient's arterial blood oxygenation and core temperature by an ear probe consisting of a housing, ear plug, two light emitting devices, one light detecting device, a heater and a temperature detector, comprising steps of
Attaching temperature sensor to a flexible ear plug;
Inserting the ear plug into the patient's ear canal;
Alternatively transmitting to the ear canal two wavelengths of light from two light emitting devices and measuring the reflected light by a light detecting device;
Measuring temperature of said ear plug by said temperature sensor;
Measuring temperature of the ear probe by said temperature detector;
Generating heat by said heater to minimize temperature difference between said temperature sensor and said temperature detector;
Computing level of blood oxygenation from the signals detected by said light detecting device, and
Computing the patient core temperature from signals received from said temperature sensor and temperature detector.

5. A method for monitoring patient's arterial blood oxygenation and core temperature by a body surface probe consisting of a housing, two light emitting devices, one light detecting device, a heater and a temperature detector, comprising steps of
Inserting the probe to the surface of a patient's body;
Alternatively transmitting to the patient body two wavelengths of light from two light emitting devices and measuring the reflected light by a light detecting device;
Measuring surface temperature of the patient by said temperature sensor;
Measuring temperature of the probe by said temperature detector;
Generating heat by said heater to minimize temperature difference between said temperature sensor and said temperature detector;
Computing level of blood oxygenation from the signals detected by said light detecting device, and
Computing the patient core temperature from signals received from said temperature sensor and temperature detector.
